(19) United States
(12) Patent Application Publication
(10) Pub. No.: US 2009/0024046 A1
(43) Pub. Date: Jan. 22, 2009

(54) Apparatus and method for detection of one lung intubation by monitoring sounds

(75) Inventors: Gabriel Gurman, Omer (IL); Noa Tejman-Yarden, Beer Sheva (IL); Joseph Tabrikian, Tel Aviv (IL); Lior Weizman, Rishon Lezion (IL); Arnon Cohen, Omer (IL); Boaz Cohen, Lehavim (IL)

Correspondence Address: EMPK & Shiloh, LLP;c/o Landon IP, Inc., 1700 Diagonal Road, Suite 450, Alexandria, VA, 22314, US

(73) Assignees: Ben Gurion University of the Negev Research and Development Authority, Beer Sheva (IL); Mor Rearch Application Ltd., Petach Tikva (IL)

(21) Appl. No.: 10/599,598

(22) PCT Filed: Apr. 3, 2005
(86) PCT No.: PCT/IL2005/000369

§ 371 Date: Aug. 7, 2008

Related U.S. Application Data
(60) Provisional application No. 60/559,993, filed on Apr. 4, 2004.

(51) Int. Cl. A61B 5/08

(52) U.S. Cl. 600/529

(57) ABSTRACT

A method and a device for the acoustic detection of a one lung intubation situation in a human subject are disclosed. According to some embodiments, the disclosed method includes computing an autoregressive moving average (ARMA) or an autoregressive function of electrical signals received from acoustic detectors placed at different locations on the body of the subject. Appropriate locations for acoustic detectors include the back region and the chest region. The disclosed method and apparatus are insensitive to uncancelled, random background noise with a loudness associated with an operating room or intensive care ward. The disclosed device is configurable so that the relative occurrence rate of missed detections or false negatives and false positive alarms can be modified. In one exemplary embodiment, the device is adapted such that at most 9% of identifications are false positive identifications, and at most 2% of identifications are false negative identifications.

### FIELD OF THE INVENTION

The present invention relates to acoustic detection of one lung intubation in ventilated patients.

### BACKGROUND OF THE INVENTION

During general anesthesia, for proper air way management, an endotracheal tube is inserted into the patient's trachea through which the patient is ventilated. The tube is inserted during the primary induction and placed so that its tip is located above the carina—the bifurcation of trachea into the two main bronchi. The location of the tip of tube is critical: it should be placed, and maintained above the bifurcation. A correct position of the tube, in which both lungs are ventilated, is called Tracheal Intubation (TRI). If the tube is misplaced or shifted due to patient movements, cases of One Lung Intubation (OLI) may occur. Prolonged cases of OLI should be avoided since it may cause insufficient oxygenation and may damage the non-ventilated lung. OLI was found to be a cause of desaturation and a cause of malfunction during anesthesia, and there is currently no reliable device or method for detecting OLI situations.

Currently known methods for detecting one lung intubation including the stethoscope and capnograph, have proven either unreliable. Pulse oximetry is the most reliable known method but provides results with latency of 2 to 5 minutes, which may be too long to prevent damage. There is an ongoing medical need for methods and devices for detecting one lung intubation in real time.

The following published documents provide potentially relevant background art and are incorporated herein by reference:
Sod-Moriah G., Gelber O., Gurman G. and Cohen A. “Monitoring of Separate Lung Ventilation in Anesthesia and Intensive Care,” Proc. of the IEEE 18th convention of electrical and electronics engineers in Israel, Tel-Aviv, Mar. 7-8, 1995.
Sod-Moriah G., Cohen A. and Gurman G., “Detection of One Lung Intubation Incidents in General Anesthesia and Intensive Care,” Proc. of the 13th Int. Conf. BIOSIGNAL 96, pp. 282-284, Brno, Czech Republic, 1996;
US 2003/0018276 of Mansy H. and Sandier R. titled “Acoustic detection of endotracheal tube location.”

It is also noted, that a poster presentation Gurman et al., “Continuous Monitoring of Separate Lung Ventilation” ASA annual Meeting Orlando Fla. Oct. 12-16, 2002 provides potentially relevant background art.

US 2003/0018276 of Mansy H. and Sandler R. titled “Acoustic detection of endotracheal tube location” purportedly disclosed a system and method for use in detecting an endotracheal tube location within a body by electronically detecting breath sounds. Unfortunately, insufficient details were provided to allow others to reproduce the disclosed results.

There is an ongoing medical need for methods and apparatus for reliably detecting a one lung intubation condition in ventilated patients. Preferably, the method and apparatus will be operative in the presence of background noise of an operating room or intensive care ward.

### SUMMARY OF THE INVENTION

It is now disclosed for the first time a method of detecting a one lung ventilation situation in a human subject. This method includes detecting indigenous lung sounds emanating from a region of the body with acoustic sensors to produce an electronic signal, and generating an output indicative of the one lung ventilation situation by processing said detected indigenous lung sounds.

According to some embodiments, the processing includes computing an autoregressive moving average (ARMA) or autoregressive model of the electronic signal.

Not wishing to be bound by theory, it is understood that the human body is not composed of a uniform medium, but is heterogeneous. Local acoustic properties vary between different types of tissue. As such, noise generated by sources such as the lungs is subjected to a certain amount of dispersion as the noise is transmitted through the human body. Parts of a specific noise generated by a source and transmitted through the body thus reach a detector on the surface of the human body at different times. Therefore, it is now disclosed that there is a correlation between a measured noise signal emanating from the lungs and the history of the measured noise signal.

As such, non-linear models or even linear models such as autoregressive moving average (ARMA) models or autoregressive models provide a reasonable representation of a source noise signal, and are useful for determining whether detecting lung sounds are from one or two intubated lungs.

In some embodiments, the disclosed method includes determining a number of active distributed noise sources of indigenous lung sounds or a number of distributed random sources in order to detect a one lung intubation situation, wherein a detection of one active distributed noise source is indicative of OLI while two active distributed noise sources indicates TRI. In some embodiments, the detection is carried out by the general approach of Blind Source Separation. Unlike previously disclosed methods for Blind Source Separation, the currently disclosed method does not require a determining of the actual signal generated by the distributed noise sources. Furthermore, the methods of the present invention are appropriate for any distributed noise source.

Furthermore, it is noted that the present invention is not limited by the aforementioned techniques, and that any method known in the art for obtaining or recovering any or part of spatial statistics of distributed noise sources from measured lung noises is appropriate for the present invention. In some embodiments, the processing of lung sounds includes using neural networks in order to obtain spatial statistics of the of the lung noises. In some embodiments, the processing of lung sounds includes linear or non-linear modeling of the lung sounds such as Hidden Markov Model (HMM). In some embodiments, the processing of lung sounds includes blind deconvolution and system identification using higher-order statistics.

It is now disclosed for the first time a method of detecting a one lung ventilation situation in a human subject. The presently disclosed method includes electronically detecting indigenous lung sounds emanating from a region of the body, and generating an output indicative of the one lung ventilation situation by processing the detected lung sounds.

According to some embodiments, the detecting includes receiving a plurality of electrical signals from a plurality of acoustic sensors, and at least one acoustic sensor is disposed adjacent to at least one region selected from the group consisting of a chest region of the body and a back region of the body. Exemplary locations for the acoustic sensors include the left side of the chest, the right side of the chest, the left side of the back and the right side of the back.

In some embodiments, the processing includes processing only electrical signals from acoustic sensors placed on the back. In some embodiments, the processing includes processing only electrical signals from acoustic sensors placed on the chest.

As noted earlier, local acoustic properties vary between different types of tissue leading to multipath propagation of lung sounds. The detected sounds can be described as a convolutive mixture and modeled by a convolutive model. As such, the detected signal can be said to have memory.

In some embodiments, the detecting includes receiving a plurality of electrical signals from a plurality of acoustic sensors, and the processing includes computing a parameter indicative of a relation between a received electrical signal and a past and/or future behavior of the received electrical signal. For embodiments where a different electrical signal is received from each acoustic sensor, it is noted this parameter may relate any of received electric signals with the past and/or future behavior of the same or any other received electric signal.

In some embodiments, the processing includes computing a parameter indicative of a relation between a received signal and at least one of a history and a future behavior of the received signal.

In some embodiments, the parameter is indicative of a relation between an electrical signal from a first acoustic sensor among the plurality of acoustic sensors, and the history and/or future behavior of the electric signal from the same first acoustic sensor.

In some embodiments, the processing includes computing a parameter indicative of a relation between a received electrical signal during a first time window and a received electrical signal during a second time window, wherein the first and second time windows may overlap.

In some embodiments, the first and second time windows overlap by at least 0.5 seconds. Alternately, the first and second time windows overlap by at least 0.75 second. Alternately, the first and second time windows overlap by at least 1 second. Alternately, the first and second time windows overlap by at least 1.5 seconds. Alternately, the first and second time windows overlap by at least 2 seconds.

It is noted that the time window overlap is an optional feature, and in some embodiments there is no overlap whatsoever between analyzed time windows.

In some embodiments, the parameter indicative of a relation between a received electrical signal and a history of a received electrical signal is computed for a plurality of times.

In some embodiments, the relation is indicative of a conditional probability relation such as a conditional probability that a future signal will have a certain form given the form of the present signal.

In some embodiments, the processing includes computing a parameter related to a covariance matrix of said conditional probability relation. In one particular embodiment, the covariance matrix is a residual covariance matrix.

One exemplary parameter indicative of a one lung ventilation situation is the magnitude of an eigenvalue of the residual covariance matrix. Selection of the specific eigenvalue for detecting one lung intubation depends on the specific number of acoustic sensors employed. In some embodiments, a lower value of the eigenvalue of the residual covariance matrix is indicative of the one lung ventilation situation.

Although certain embodiments of the present invention relate to techniques of Blind Source Separation, recovery of original transmitted lung sounds is not a requirement of the present invention. According to some embodiments, the processing includes determining only a number of distributed noise sources of said indigenous lung sounds.

Nevertheless, in some embodiments an estimate of the originally transmitted lung sounds is obtained.

According to some embodiments, the processing includes obtaining a parameter indicative of spatial statistics of the indigenous lung sounds.

In some embodiments, the spatial and/or temporal statistics of the indigenous lung sounds is monitored in time, and deviations in the spatial statistics of the lung sound are indicative of a change in intubation status, e.g. a change from TRI to OLI. In one particular example, the spatial statistics of the lung sounds are obtained during a time period of known TRI such as at the beginning of surgery to train the system. At a later time for which it is desired to detect the presence or absence of the one lung ventilation situation, spatial statistics of the lung sounds are compared with their value during the time period of known TRI.

According to some embodiments, the processing includes determining a source scattering parameter indicative of a scattering (spatial distribution) of noise sources. More scattered, non-coherent point noise sources are more indicative of TRI, while less scattered noise sources are evident by a smaller second eigenvalue of the residual covariance matrix.

In experiments conducted by the present inventors, a one lung ventilation situation in a human subject was determined in the presence of uncancelled, random background noise associated with an operating room or intensive care ward. Although certain types of background noise can be cancelled out using techniques such as simple filtering or adaptive noise cancellation, and the presently disclosed methods and devices do not preclude usage of these techniques, not every random background noise is necessarily cancelled out, especially if this noise is not treated using the aforementioned techniques. The present invention provides methods and devices for detecting a one lung ventilation situation even in the presence of uncancelled, random background noise of a loudness associated with an operating room or intensive care ward.

In some embodiments, the processing includes processing the detected indigenous lung sounds in a way that is insensitive to uncancelled, random background noise of a loudness associated with an operating room.

In some embodiments, uncancelled random background noise includes at least 70 decibels of uncancelled noise, or at least 75 decibels of uncancelled noise, or at least 80 decibels of uncancelled noise.

Optionally, the stage of detecting includes detecting noise other than lung sounds, and the stage of processing includes using an adaptive filtering technique to filter noise.

In some embodiments, a specific parameter indicative of a one lung intubation situation is calculated, and when the value of the calculated parameter drops below or climbs above the threshold of a predetermined threshold value, output indicative of a one lung intubation situation is generated. Thus, by adjusting this predetermined threshold value, it is possible to configure the device such that an occurrence rate of false alarms or false positives and missed OLI situations or false negatives changes according an error tradeoff curve.

For example, in some embodiments, a smaller value of the calculated parameter is indicative of a one lung intubation situation. Thus, by raising the predetermined threshold value, instances of one lung intubation are less likely to be missed, and the occurrence rate of false negatives drops, while concomitantly, the device is more likely to generate false alarms or false positives.

Thus, in some embodiments, the processing unit is adapted such that at most 9% of identifications of OLI are false positive identifications, and at most 2% of said identifications are false negative identifications.

Alternately, the processing unit is adapted such that at most 4.5% of identifications of OLI are false positive identifications, and at most 4.5% of said identifications are false negative identifications.

It is now disclosed for the first time a method including selecting a population of human subjects sufficiently large to give statistically significant results, and identifying a one lung intubation situation in a subpopulation of the population, wherein at most 9.6% of the identifications are misidentifications.

It is now disclosed for the first time a method including selecting a population of human subjects sufficiently large to give statistically significant results, and identifying a one lung intubation situation in a subpopulation of the population, wherein at most 4.8% of the identifications are false positive identifications, and at most 4.8% of the identifications are false negative identifications.

It is now disclosed for the first time a method including selecting a population of human subjects sufficiently large to give statistically significant results, and identifying a one lung intubation situation in a subpopulation of the population, wherein at most 9% of the identifications are false positive identifications, and at most 2% of the identifications are false negative identifications.

In some embodiments, a population sufficiently large to give statistically significant results includes at least 20 individuals. Alternately, the population includes at least 50 individuals. Alternately, the population includes at least 200 individuals. Alternately, the population includes at least 1000 individuals.

These and further embodiments will be apparent from the detailed description and examples that follow.

### BRIEF DESCRIPTION OF THE FIGURES

FIG. 1 provides block diagram of an exemplary MIMO AR model.

FIG. 2 provides a graph of Pₑ of GLRT for coherent sources.

FIG. 3 provides a graph of eigenvalues of \hat{R} versus the scattering level, ε.

FIG. 4 provides a schematic of exemplary locations of microphones on the back of the patient according to some embodiments of the present invention. The TRI situation is illustrated in FIG. 4.

FIG. 5 illustrates some recorded breathing cycles both for both one lung intubation and tracheal intubation cases.

FIG. 6 provides an exemplary graph of the second largest eigenvalue of \hat{R} as a function of time for both one lung intubation and tracheal intubation cases.

FIG. 7 provides the DET of a classifier based on the second highest eigenvalue of estimated \hat{R}.

### DETAILED DESCRIPTION OF THE INVENTION

It has been discovered in accordance with some embodiments of the present invention that computing certain autoregression functions of a detected acoustic signal enables detection of a one lung intubation situation, even in the presence of background noise associated with operating rooms and intensive care wards. In particular, an algorithm for detecting the number of ventilated lungs from recorded breathing sounds has been developed. In some embodiments, this algorithm assumes a MIMO (Multiple Input Multiple Output) system, in which a multi-dimensional AR (Auto-Regressive) model relates the input (lungs) and the output (recorded sounds). The unknown AR parameters are estimated, and a detector based on the estimated eigenvalues of the residual covariance matrix is developed, in order to detect a one lung ventilation situation.

In the examples presented, a number of noise sources is estimated using measures such as the Akaike Information Criterion (AIC) or the Minimum Description Length (MDL). All of these measures make several assumptions about the sources. Unfortunately, the problem at hand does not obey these assumptions, the most notorious of which is the assumption of coherent distributed noise source. The lung is a diffused source rather than a point source.

Under the assumption of coherent distributed sources the large eigenvalues of the residual covariance matrix correspond to the sources and the small ones correspond to the diffuse noise. In the case of diffuse (distributed), non-coherent sources, the distinction between these two groups is not clear. Threshold methods are used in order to estimate the number of sources.

Although in some embodiments this algorithm derives from a Blind Source Separation model, a presently-disclosed algorithm estimates only the number of active sources or lungs, and does not require estimation of the source signal itself. The source signal is transmitted via the chest or back of the patient to the sensor. Although there are non-linearities associated with transmission channel, it has been discovered by the present inventors that assuming a linear transmission channel is functional for detecting one lung intubation. Furthermore, although the most general discretion of a linear channel is an ARMA (Auto Regressive Moving Average) with poles and zeros, it is known that a less general high-order AR (Auto Regressive) model including only poles provides an approximation to an ARMA model. In accordance with some embodiments of the present invention, it is disclosed that the AR model is functional for detecting one lung intubation.

The methods of the present invention are appropriate for any distributed noise source. In some embodiments, a “distributed noise source” as used herein is composed of point noise sources distributed in an area of at least 75 cm². In some embodiments, a “distributed noise source” as used herein is composed of point noise sources distributed in an area of at least 200 cm². In some embodiments, a “distributed noise source” as used herein is composed of point noise sources distributed in an area of at least 400 cm².

According to some embodiments, spatial and/or temporal statistics of indigenous lung sounds are computed. Examples of computed spatial include but are not limited to a cross/joint covariance matrix or cross/joint spectrum between the processes at different sensors. Alternatively, it can be the cross/joint cumulant of any order or cross/joint higher-order spectra of any order greater than two between the processes of different sensors. Alternatively, it can be joint probability density function of the measurement processes at the different sensors.

The following examples are to be considered merely as illustrative and non-limiting in nature. It will be apparent to one skilled in the art to which the present invention pertains that many modifications, permutations, and variations may be made without departing from the scope of the invention.

### EXAMPLES

### Example 1

### Model Formulation

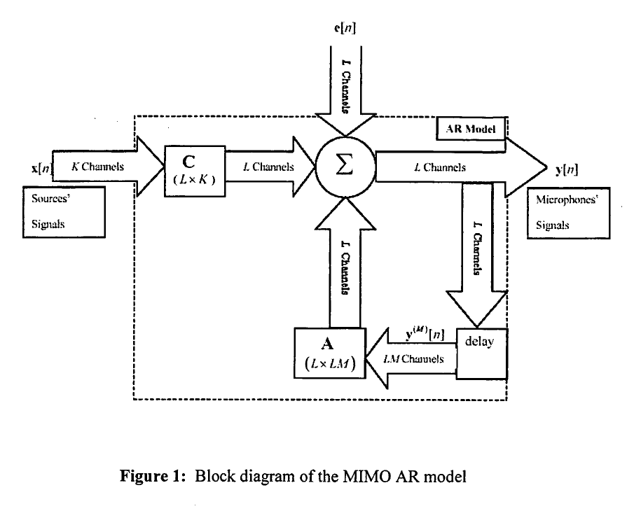

In the present example, the breathing sound signals are recorded by 4 microphones attached to the patient's back. Previous attempts to detect OLI by comparing the amplitude of the recorded sounds in right and left sides did not result in reliable methods, because each one of the microphones records sounds generated by both lungs. In order to overcome this problem, a convolutive mixture model approach is presented. In the current examples, an AR model that relates the lungs and the microphones is assumed. The AR model was chosen because it is commonly used in applications of speech and audio processing and its computational complexity is relatively simple. In this model, each ventilated lung represents a source. Our goal is to detect a situation of which only one lung is ventilated, from the received signals by the sensors. It is assumed that the signals generated by the ventilated lungs are independent. FIG. 1 shows a block diagram of the proposed MIMO-AR model, in which x[n] represents the sources (lungs), and y[n] represents the sensor (microphones) measurements.

Let K and L denote the number of sources (lungs) and sensors (microphones), respectively (K<L). Therefore, the vector of source signals, x[n], is defined as a K×1 vector as follows:

x[n]=[x₁[n]x₂[n] . . . x_{K}[n]]^{T}. (1)

The L×1 measurement vector is defined as:

y[n]=[y₁[n]y₂[n] • • • y_{L}[n]]^{T} (2)

The relation between the source signals and the measurements is assumed to be given by a MIMO-AR model:

y[n]=Ay^{(M)}[n]+Cx[n]+e[n], (3)

where y^{(M)}[n] is an ML×1 vector defined as follows:

y^{(M)}[n]=[y₁^{(M)}^{^{T}}[n]y₂^{(M)}^{^{T}}[n] • • •]y_{L}^{(M)}^{^{T}}[n]]^{T} (4)

and yᵢ^{(M)}[n] is an M×1 vector which contains the past values of the i-th sensor, yᵢ[n], up to sample M:

yᵢ^{(M)}[n]=[yᵢ[n−1]yᵢ[n−2] • • • yᵢ[n−M]]^{T}. (5)

A is an L×ML matrix defined as:

$$A = [ \begin{matrix}{a}_{11}^{T} & ⋯ & {a}_{1 L}^{T} \\ ⋮ & & ⋮ \\ {a}_{L 1}^{T} & ⋯ & {a}_{LL}^{T}\end{matrix} ] , \qquad ( 6 )$$

where aᵢⱼ is an M×1 vector, which relates the samples of the i-th sensor, yᵢ[n], with the past values of the j-th sensor, yⱼ[n−1], . . . , yⱼ[n−M]. C is an L×K matrix whose i,j-th element relates the samples of source j and sensor i. Finally, e[n] is an L×1 vector representing additive white noise. It is assumed that the noise and source signals are independent, zero-mean, Gaussian with covariance matrices σ²I and I, respectively. The last assumption can be employed with no loss of generality, because the covariance of the sources is determined by the matrix C, as it can clearly be seen from (3). As a result, it is obtained that the conditional distribution of y[n]|y^{(M)}[n] is Gaussian: y[n]|y^{(M)}[n]˜N(Ay^{(M)}[n], R), where R is defined as:

R=CC^{T}+σ²I. (7)

It is noted that the unknown parameters: A, R, M and K must be estimated from a set of N measurements, y[1], . . . , y[N]. It is also assumed that all the initial conditions are zero, i.e. e[n],x[n]=0 for n<0, and that the input and noise signals are stationary. In fact, successful estimation of K, the number of sources (lungs), is the key for the OLI detection.

### Example 2

### The ML Estimator

In order to determine the number of sources, K, we need first to estimate the unknown matrices, A and R, from the N samples of the data: y[1], . . . , y[N]. For this purpose, the Maximum-Likelihood (ML) estimator is used. The ML estimator of the matrices A and R, is obtained by maximizing the logarithm of the conditional probability density function (pdf) of the output samples given the unknown matrices, which is:

$$log f ( y [ 1 ] , … , y [ N ] \vert R , A ) = - \frac{NL}{2} log ( 2 π ) - \frac{N}{2} log \vert R \vert -- \frac{1}{2} {\sum }_{n = 1}^{N} [ {( y [ n ] - {Ay}^{( M )} [ n ] )}^{T} {R}^{- 1} ( y [ n ] - {Ay}^{( M )} [ n ] ) ] . \qquad ( 8 )$$

The log-likelihood function can be maximized by equating its derivatives with respect to A and R, and solving the two resulting matrix equations. This process yields (Proof: See Appendix A):

$$\begin{matrix}{\hat{A}}_{ML} = ( {\sum }_{n = 1}^{N} y [ n ] {u}^{T} [ n ] ) {( {\sum }_{n = 1}^{N} u [ n ] {u}^{T} [ n ] )}^{- 1} \\ and & ( 9 a ) \\ {\hat{R}}_{ML} = \frac{1}{N} {\sum }_{n = 1}^{N} y [ n ] {y}^{T} [ n ] - {\hat{A}}_{ML} ( \frac{1}{N} {\sum }_{n = 1}^{N} u [ n ] {y}^{T} [ n ] ) & ( 9 b )\end{matrix}$$

The use of model order selection methods based on information theoretic criteria [11]-[14] seems to be the natural method in order to estimate the model order, M, and the number of sources, K. This method was developed and tested during our work, but did not show a reliable result when applied to real breathing sound signals. Therefore, a Generalized Likelihood Ratio Test based method was developed and tested as shown in the next Section.

### Example 3

### Generalized Likelihood Ratio Test

In the private case of lungs as sources, the number of sources can be only one or two. Therefore, for the purpose of decision of between TRI case and OLI case, the GLRT is used [15]. This test is based on the ratio between the probability density function under each hypothesis, while the maximum likelihood estimator is used to estimate the unknown parameters under each hypothesis. Let us denote the following hypothesis:

H₁: Only one source exists for the system (OLI case, K=1)

H₂: There are two sources for the system (TRI case, K=2)

The development of the Log-likelihood function under the i-th hypothesis, leads to the following expression (assuming the noise variance, σ², is known):

$$log f ( y [ 1 ] , … , y [ N ] \vert R , A ; {H}_{i} ) = - \frac{NL}{2} log ( 2 π ) - \frac{N}{2} log ( {\prod }_{i = 1}^{K} {{l}_{i} ( {σ}^{2} )}^{L - K} ) - \frac{NL}{2} \qquad ( 10 )$$

where {lᵢ}ᵢ₌₁² are the two highest eigenvalues of \hat{R} (l₁≧l₂), and L is the number of sensors (Proof: See Appendix B).
As a result, the GLRT for decision between H₁ and H₂ is as follows:

$$log [ \frac{f ( y [ 1 ] , … , y [ N ] \vert R , A ; {H}_{1} )}{f ( y [ 1 ] , … , y [ N ] \vert R , A ; {H}_{2} )} ] = \frac{N}{2} log ( \frac{{l}_{1} \cdot {{l}_{2} ( {σ}^{2} )}^{L - 2}}{{{l}_{1} ( {σ}^{2} )}^{L - 1}} ) = \frac{N}{2} log ( \frac{{l}_{2}}{{σ}^{2}} ) \qquad ( 11 )$$

As can clearly be seen from (11), the second highest eigenvalues of \hat{R} is actually the detector of OLI situation, under the assumption that σ² is known, and that the sources are point sources. Simulation results given in the next section show the performance of the proposed detector under coherent and incoherent distributed sources assumption, while the last assumption is a more accurate model for lungs sources.

### Example 4

### Simulation Results

### 1. Coherent Sources

In order to evaluate the performance of the estimators as a function of the parameters of the model, simulations with synthetic data were performed. The MIMO-AR system as defined in (3), was simulated and the ML estimators of R and A were calculated according to (9). In the simulations the parameters of the system were as follows (unless otherwise is indicated): the number of sources was K=2, the number of sensors was L=4 with AR order, M=5 and noise variance, σ²=1. The matrix C was chosen to be:

$$C = [ \begin{matrix}0.3 & 0.15 \\ 0.15 & 0.3 \\ 0.9 & 0.6 \\ 0.6 & 0.9\end{matrix} ]$$

and the matrix A was chosen to be stable representing system poles inside the unit circle. Simulation results of the ML estimators of the matrices A and R, can be found in.

The behavior of the GLRT as a function of the number of independent samples, N, is examined. The second highest eigenvalue, l₂, was extracted and compared to a threshold value of the noise level, σ². A total number of J=1000 iterations for each N were performed, and the probability of error of K is defined as:

$${P}_{e} = \frac{number of uncorrectly estimated K}{J} \qquad ( 12 )$$

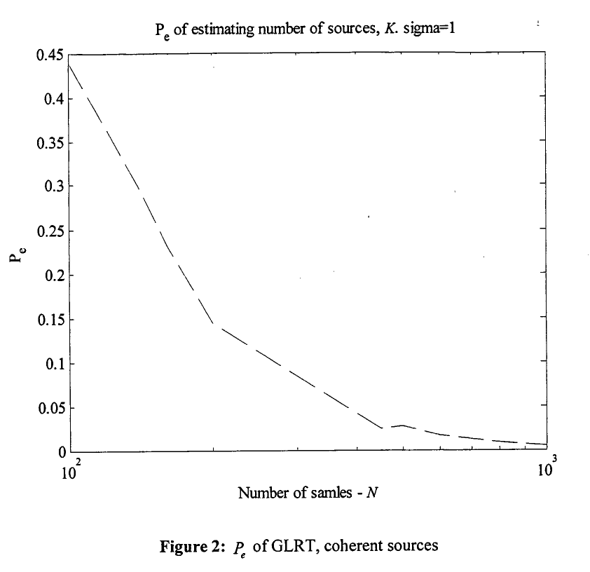

FIG. 2 shows the probability of error, Pₑ, as function of number of samples, N. It can be seen that probability of error decrease as the number of samples grows. This fact justifies the use of a threshold value of the noise level when the sources are coherent.

### 2. Incoherent Distributed Sources Simulation Results

It is well known that each lung is composed of several independent point sources, and therefore should be treated as distributed sources. In order to evaluate the performance of the algorithm under this assumption, spatially distributed sources were synthesized. In this simulation, two distributed sources were synthesized. Each distributed source was composed of four independent point sources with close spatial signatures. Therefore, the columns of C were chosen to be:

$$C = [ \begin{matrix}{\underbrace{\begin{matrix}{c}_{1} & {c}_{1} + ɛΔ {c}_{11} & {c}_{1} + 2 ɛΔ {c}_{12} & {c}_{1} + 3 ɛΔ {c}_{13}\end{matrix}}}_{source 1} & {\underbrace{\begin{matrix}{c}_{2} & {c}_{2} + 2 ɛΔ {c}_{21} & {c}_{2} + 3 ɛΔ {c}_{22} & {c}_{2} + ɛΔ {c}_{23}\end{matrix}}}_{source 2}\end{matrix} ] \qquad ( 13 )$$

where c₁ and c₂ are orthogonal, and {cᵢ Δcᵢ₁ Δcᵢ₂ Δcᵢ₃} is an orthonormal group. ε is a constant which determines the distribution width. The vector of sources x[n] represents eight independent sources. Therefore, the product Cx[n] represents two distributed sources, where each one is constructed by 4 independent sources.

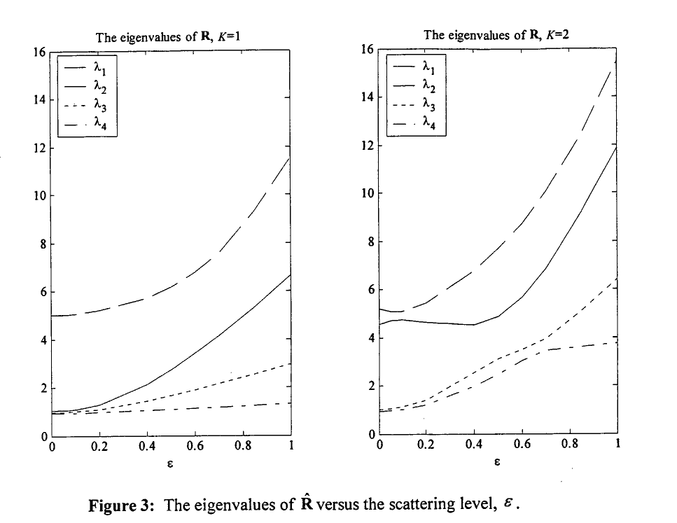

In order to examine the performance of the GLRT under incoherent distributed sources assumption, a comparison between the eigenvalues of \hat{R} for cases of one and two distributed sources is given. In FIG. 3 the eigenvalues of \hat{R} are drawn as function of ε for K=1 and K=2. It can be seen from FIG. 3 that even when the sources are widely distributed, the eigenvalues of the source signals sub-space are separated from the eigenvalues of the noise sub-space. Therefore, despite the fact that the lungs do not function as a coherent sources model, it was decided to examine the second highest eigenvalue of \hat{R} as a detector for OLI situation in real breathing sound signals. In addition, it can be also be seen the threshold value in the case where the sources are widely distributed should be higher than the noise level, σ²=1.

### Example 5

### Experimental Results

In order to examine the disclosed model for OLI detection, a database of recorded breathings was established. The database was composed of 24 patients which were recorded in a surgery room in both situations: during correct ventilation, when the tip of the tube is placed above the carina, and during a situation of OLI when the tip of the tube is under the carina and only one lung is ventilated.

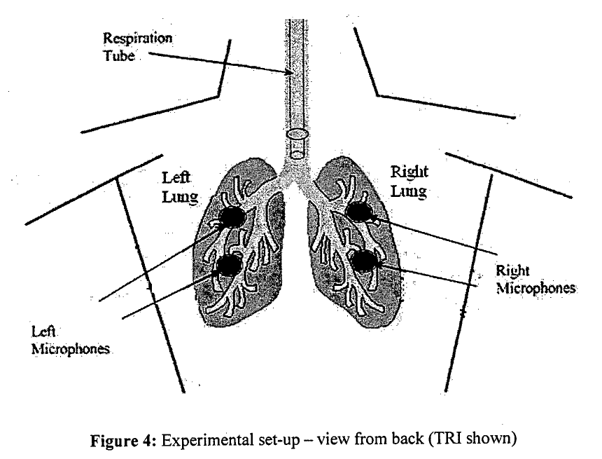

During each experiment, the microphones were attached to the patient's back, as shown in FIG. 4, recorded the breathing sounds of the patients in both situations. The ventilations were performed manually and not mechanically, in order to achieve higher signal-to-noise ratio in the recorded sounds, and the real position of the tube was validated each time by fiber-optic. The experiments were performed in the main surgery room of medical center Soroka—Israel, during the anesthesia part in the beginning of the surgeries.

In order to attenuate some of the irregular background noises outside of the spectral range for breathing signals, such as monitor beeps, doctors' discussions, etc. the recorded signals were band-pass filtered by a Butterworth filter with a bandwidth of 100 Hz-600 Hz. The recorded sounds were sampled at 4 kHz.

Because of the cut-off frequency of 600 Hz, down-sampling operation with a factor of 0.3 was performed.

The signal amplitude from each microphone depends on the particular location of the microphone on patient's body, the anatomy of the particular patient, and on the gain of the sampling system. Therefore, it turned out that each channel's output had a different signal amplitude, and the noise variance, σ², also differed between microphones. In order to treat this problem, a normalization of each channel according to the noise level on the channel was done.

It is also noted that the aforementioned techniques were only sufficient to reduce some ambient noise associated with an operating room, and the algorithm itself was robust enough to determine a OLI situation in a manner that was insensitive to irregular noise of an operating room.

The recorded breathing signals contain both situations of OLI and TRI. The breathing signals were limited to cut-off frequency of 4 kHz, and the data were divided into windows of 2000 samples each, with 80% overlap. Because of fact that AIC and MDL have chosen the highest available AR model order when applied to real data, an arbitrary AR order of 15 was set considering the computation complexity and the available processing time. The unknown matrices A and R were estimated for each window, using the ML estimator developed in Section II.

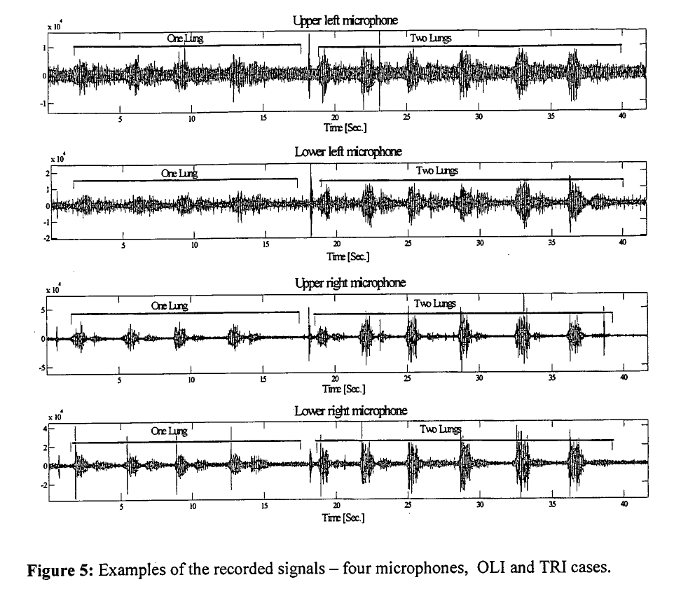

FIG. 5 shows a few breathing cycles of both OLI and TRI situations, recorded by the four microphones after pre-processing. As it can be seen from this figure, determination between OLI and TRI cases by only the amplitude of the recorded sounds is not a simple task.

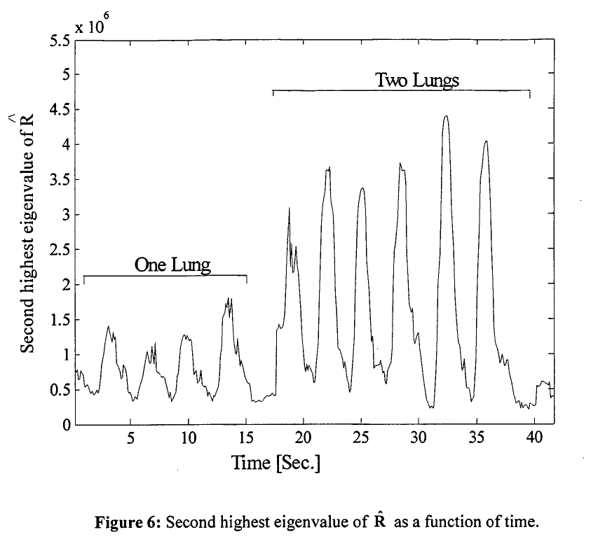

FIG. 6 shows the second highest eigenvalue of \hat{R}_{ML} as a function of time, as a result of processing the measurements shown in FIG. 5. As it can clearly be seen from FIG. 6, OLI and TRI cases can clearly be discriminated, by the second highest eigenvalue of \hat{R}_{ML} in every breathing cycle. The results of the proposed algorithm were consistent over the 24 experiments.

Estimation of the performance of the system was performed using “Leave some out method” as follows. Twenty different experiments were used to extract the histograms in order to train the system. The rest of the 4 experiments were used to validate the system and were tested according to the extracted statistics in the training process. This process was repeated 6 times, each time a different group of four validation experiment was used. As a result, a validation was performed using a total number of 24 experiments, in a “patient independent” mode.

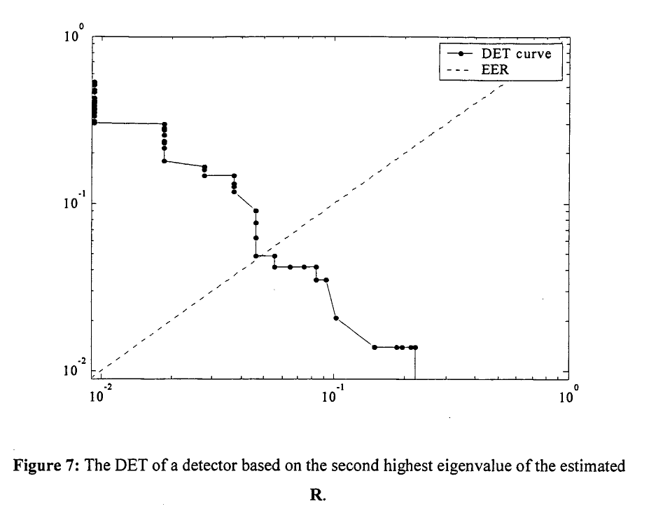

There are two types of errors in OLI detection: Pₘᵢₛₛ, the probability of a true OLI to be wrongly detected as TRI, and P_{FA}, the probability of TRI to be detected as OLI. The Detection Error Tradeoff (DET) curve is a common mean to display these errors. The DET curve provides information about the device's performance, where each point on the curve shows the P_{FA} and Pₘᵢₛₛ for a given threshold. The threshold of a real monitoring system should be calculated according to the requested sensitivity of the system, while taking into consideration the allowed Pₘᵢₛₛ of the system. FIG. 7 shows the DET curve of the proposed decision system, which was computed according to the 6 iterations described above. The Equal Error Rate (EER) point is defined as the point on the DET curve where Pₘᵢₛₛ=P_{FA}, is 4.8. Naturally, more importance should be given to Pₘᵢₛₛ rather than to P_{FA}. Therefore, it is assumed that in a practical system the selected activity point on the DET curve will be where Pₘᵢₛₛ=2% and P_{FA}=9%.

### Example 6

### Discussion

From the practical point of view, these examples have illustrated methods and apparatus for detection of OLI. An algorithm for detection of OLI by monitoring lungs sounds was developed. In order to examine the algorithm performance, a database of recorded breathing sound signals of patients during OLI and TRI situations was established. It has been shown that assuming a MIMO-AR model and selecting the second highest eigenvalue of the residual covariance matrix as a feature proves itself as a reliable method for detection of OLI on real breathing sound signals.

Because of the fact that a pre-processing according to the surgery conditions has to be performed, it is disclosed that optional automatic training of the system before every surgery in order to enable it to set the optimal gain for each microphone is advantageous.

### APPENDIX A

### Proof of Equation (9)

Maximization of (8) with respect to the unknown parameters, A,R, is achieved via equating the corresponding partial derivatives to zero. Only the last term of (8) which is:

$$- \frac{1}{2} {\sum }_{n = 1}^{N} [ {( y [ n ] - {Ay}^{( M )} [ n ] )}^{T} {R}^{- 1} ( y [ n ] - {Ay}^{( M )} [ n ] ) ] == - \frac{1}{2} {\sum }_{n = 1}^{N} [ \begin{matrix}{y}^{T} [ n ] {R}^{- 1} y [ n ] - {y}^{( M ) T} [ n ] {A}^{T} {R}^{- 1} {y}^{( M )} [ n ] - \\ {y}^{T} [ n ] {R}^{- 1} {Ay}^{( M )} [ n ] + {( {Ay}^{( M )} [ n ] )}^{T} {R}^{- 1} ( {Ay}^{( M )} [ n ] )\end{matrix} ] \qquad ( 14 )$$

is relevant to calculate the derivative of the log-likelihood with respect to A. The derivative of a scalar a with respect to a matrix B is defined as:

$$\frac{\partial a}{\partial B} = [ \begin{matrix}\frac{\partial a}{\partial {b}_{11}} & ⋯ & \frac{\partial a}{\partial {b}_{1 N}} \\ ⋮ & & ⋮ \\ \frac{\partial a}{\partial {b}_{M 1}} & ⋯ & \frac{\partial a}{\partial {b}_{MN}}\end{matrix} ] , \\ where$$

$$B = [ \begin{matrix}{b}_{11} & ⋯ & {b}_{1 N} \\ ⋮ & & ⋮ \\ {b}_{M 1} & ⋯ & {b}_{MN}\end{matrix} ] .$$

We shall use the following identities:

$$\begin{matrix}\frac{\partial }{\partial A} [ \begin{matrix}{x}^{T} & Ay\end{matrix} ] = \frac{\partial }{\partial A} [ \begin{matrix}{y}^{T} & {A}^{T} & x\end{matrix} ] = {xy}^{T} & ( 15 ) \\ \frac{\partial }{\partial A} [ \begin{matrix}{( Ax )}^{T} & C ( Ax )\end{matrix} ] = ( C + {C}^{T} ) ( Ax ) {x}^{T} . & ( 16 )\end{matrix}$$

If A is a square matrix and f(A) is a scalar function, then:

$$\frac{\partial log f ( A )}{\partial A} = \frac{1}{f ( A )} \frac{\partial f ( A )}{\partial A} . \qquad ( 17 )$$

If A is a square and invertible matrix, then:

$$\frac{\partial \vert A \vert}{A} = \vert A \vert {( {A}^{- 1} )}^{T} , \qquad ( 18 )$$

and we obtain:

$$\begin{matrix}\frac{\partial }{\partial A} log ( f ( y [ 1 ] , … , [ N ] \vert R , A ) = - \frac{1}{2} {\sum }_{n = 1}^{N} [ - {R}^{- 1} y [ n ] {y}^{( M ) T} [ n ] - {R}^{- T} y [ n ] {y}^{( M ) T} [ n ] + ( {R}^{- 1} + {R}^{- T} ) {Ay}^{( M )} [ n ] {y}^{( M ) T} [ n ] ] , & ( 19 ) \\ \frac{\partial }{\partial {R}^{- 1}} log ( f ( y [ 1 ] , … , y [ N ] \vert R , A ) == \frac{N}{2} [ \frac{1}{\vert {R}^{- 1} \vert} \vert {R}^{- 1} \vert ( {R}^{T} ) ] - \frac{1}{2} {\sum }_{n = 1}^{N} [ \begin{matrix}y [ n ] {y}^{T} [ n ] - {Ay}^{( M )} [ n ] {y}^{T} [ n ] - \\ y [ n ] {y}^{( M ) T} [ n ] {A}^{T} + {Ay}^{( M )} [ n ] {y}^{( M ) T} [ n ] {A}^{T}\end{matrix} ] & ( 20 )\end{matrix}$$

In order to find the ML estimator of A and R, the above derivatives should be equated to zero. Since R is a covariance matrix then, R=R^{T} and R⁻¹=R^{-T}. Therefore we obtain two matrix equations with two unknown matrix variables, A and R⁻¹:

$$\begin{matrix}- \frac{1}{2} {\sum }_{n = 1}^{N} [ - {R}^{- 1} y [ n ] {y}^{( M ) T} [ n ] - {R}^{- 1} y [ n ] {y}^{( M ) T} [ n ] + 2 {R}^{- 1} {Ay}^{( M )} [ n ] {y}^{( M ) T} [ n ] ] = 0 & ( 21 ) \\ \frac{N}{2} R - \frac{1}{2} {\sum }_{n = 1}^{N} [ \begin{matrix}y [ n ] {y}^{T} [ n ] - {Ay}^{( M )} [ n ] {y}^{T} [ n ] - y [ n ] {y}^{( M ) T} [ n ] {A}^{T} + \\ {Ay}^{( M )} [ n ] {y}^{( M ) T} [ n ] {A}^{T}\end{matrix} ] = 0 & ( 22 )\end{matrix}$$

Eq. (22) can be simplified to:

$$N \hat{R} = {\sum }_{n = 1}^{N} y [ n ] {y}^{T} [ n ] - \hat{A} {\sum }_{n = 1}^{N} {y}^{( M )} [ n ] {y}^{T} [ n ] - ( {\sum }_{n = 1}^{N} y [ n ] {y}^{( M ) T} [ n ] ) {\hat{A}}^{T} ++ \hat{A} ( {\sum }_{n = 1}^{N} {y}^{( M )} [ n ] {y}^{( M ) T} [ n ] ) {\hat{A}}^{T} . \qquad ( 23 )$$

Extraction of A from (21) while assuming the matrix

$${\sum }_{n = 1}^{N} {y}^{( M )} [ n ] {y}^{( M ) T} [ n ]$$

is invertible, leads to (9a). Substituting (9a) into the last term of (23), and Extraction of R leads to (9b).

### APPENDIX B

### Proof of Equation (10)

Substituting Â and \hat{R} into (8) leads to (24):

$$log [ f ( y [ 1 ] , … , y [ N ] \vert R , A ) ] = - \frac{NL}{2} log ( 2 π ) - \frac{N}{2} log \vert \hat{R} \vert -- \frac{1}{2} {\sum }_{n = 1}^{N} [ {( y [ n ] - \hat{A} u [ n ] )}^{T} {\hat{R}}^{- 1} ( y [ n ] - \hat{A} u [ n ] ) ] \qquad ( 24 )$$

The sum term in (24) is a scalar, and therefore the trace operation can be performed on it:

$$\begin{matrix}\begin{matrix}tr ( {\sum }_{n = 1}^{N} \{ {( y [ n ] - \hat{A} u [ n ] )}^{T} {\hat{R}}^{- 1} ( y [ n ] - \hat{A} u [ n ] ) \} ) = {\sum }_{n = 1}^{N} tr \{ {( y [ n ] - \hat{A} u [ n ] )}^{T} {\hat{R}}^{- 1} ( y [ n ] - \hat{A} u [ n ] ) \} \\ = {\sum }_{n = 1}^{N} tr \{ {\hat{R}}^{- 1} ( y [ n ] - \hat{A} u [ n ] ) {( y [ n ] - \hat{A} u [ n ] )}^{T} \} \\ = tr ( {\sum }_{n = 1}^{N} \{ {\hat{R}}^{- 1} ( y [ n ] - \hat{A} u [ n ] ) {( y [ n ] - \hat{A} u [ n ] )}^{T} \} ) \\ = tr ( {\hat{R}}^{- 1} {\sum }_{n = 1}^{N} \{ ( y [ n ] - \hat{A} u [ n ] ) {( y [ n ] - \hat{A} u [ n ] )}^{T} \} ) \\ = tr ( N {\hat{R}}^{- 1} \hat{R} ) \\ = tr ( NI ) \\ = NL\end{matrix} & ( 25 )\end{matrix}$$

The fifth equality in (25) arises by substituting (9b).
The determinant of matrix is the product of its eigenvalues. Therefore, recalling {lᵢ}ᵢ₌ₗ^{L} the eigenvalues of \hat{R} in descending order (l₁≧l₂≧ • • • ≧l_{L}), |\hat{R}| can be simplified into:

$$\vert \hat{R} \vert = {\prod }_{i = 1}^{L} {l}_{i} \qquad ( 26 )$$

As appears in [16], the smallest L-K eigenvalues of \hat{R} are used to estimate σ². Therefore, (26) turns into:

$$\vert \hat{R} \vert = {\prod }_{i = 1}^{K} {{l}_{i} ( \frac{1}{L - K} {\sum }_{i = K + 1}^{L} {l}_{i} )}^{L - K} \qquad ( 27 )$$

Because of the assumption that σ² is known, it shall be substituted instead of its estimation in (27), and therefore (27) turns into:

$$\vert \hat{R} \vert = {\prod }_{i = 1}^{K} {{l}_{i} ( {σ}^{2} )}^{L - K} \qquad ( 28 )$$

Substituting (25) and (28) into (24) leads to (10).

### REFERENCES

[1] R. K. Webb, J. H. van der Walt, W. B. Runciman, J. A. Williamson et. al., “Which monitor? An analysis of 2000 incident reports,” Anesthesia and Intensive Care, vol. 21, pp. 529-542, 1993.
[2] W. B. Runciman, R. K. Webb, L. Barker and M. Currie, “The pulse oximeter: applications and limitations—An analysis of 2000 Incident Reports,” Anesthesia and Intensive Care, vol. 21, pp. 543-550, 1993.
[3] T. A. Webster, “Now that we have pulse oximeters and capnographs, we don't need precordial and esophageal stethoscope,” J. of Clinical Monitoring, vol. 3, pp. 191-192, 1987.
[4] A. Cohen and D. Landsberg, “Analysis and automatic classification of breathing sounds,” IEEE Trans. vol. BME-31, pp. 585-590, 1984.
[5] A. Cohen and A. Berstein, “Acoustic transmission of the respiratory system using speech stimulation,” IEEE Trans. on Biomedical Engineering, vol. 38, pp. 126-132, 1991.
[6] G. Sod-Moriah, A. Cohen and G. Gurman, “Detection of one lung intubation incidents in general anesthesia and intensive care,” Proc. of the 13th Int. Conf. BIOSIGNAL 96, pp. 282-284, Brno, Czech Republic, 1996.
[7] A. David, T. Lazmi, Detection of one-lung intubation, Final Project Report, Ben-Gurion University of the Negev, Department of Electrical and Computer Engineering, Beer-Sheva, Israel, August 2003.
[8] G. R. Wodicka, H. L. Golub, “A model of acoustic transmission in the respiratory system,” IEEE Trans. on Biomedical Engineering, vol. 36, No. 9, pp. 925-934, September 1989.
[9] V. K. Iyer, P. A. Rammoorthy and Y. Ploysongsang, “Autoregressive modeling of lung sounds: characterization of source and transmission,” IEEE Trans. on Biomedical Engineering, vol. 36, No. 11, pp. 1133-1137, November 1989.
[11] H. Akiake, “A new look at the statistical model identification,” IEEE Trans. Automat. Contr. vol. AC-19, pp. 716-723, 1974.
[12] H. Akiake, “Information theory and an extension of the maximum likelihood principle,” Proc. 2^{nd} Int. Symp. Inform. Theory, Suppl. Problems of Control an Inform. Theory-1973, pp. 267-281, 1973.
[13] G. Schwartz, “Estimating the dimension of a model,” Ann. Stat., vol. 6, pp. 461-464, 1978.
[14] J. Rissanen, “Modeling by shortest data description,” Automatica, vol. 14, pp. 465-471, 1978.
[15] S. Kay, “Fundamentals of Statistical Signal Processing, Volume 2”, Prentice Hall, 1998.
[16] M. Wax and T. Kailath, “Detection of signals by information theoretic criteria,” IEEE Trans. Acoust. Speech, Signal Processing, vol. ASSP-33, pp. 387-392, 1985.

While the present invention has been particularly described, persons skilled in the art will appreciate that many variations and modifications can be made. Therefore, the invention is not to be construed as restricted to the particularly described embodiments, rather the scope, spirit and concept of the invention will be more readily understood by reference to the claims which follow.

1-33. (canceled)

34) A method of detecting a one lung ventilation situation in a human subject, the method comprising:
a) electronically detecting indigenous lung sounds emanating from a region of the body with an acoustic sensors; and
b) generating an output indicative of the one lung ventilation situation by processing said detected indigenous lung sounds.

35) The method of claim 34 wherein said detecting includes receiving a plurality of electrical signals from a plurality of acoustic sensors, wherein at least one said acoustic sensor is disposed adjacent to a chest region of the body.

36) The method of claim 34 wherein said detecting includes receiving a plurality of electrical signals from a plurality of acoustic sensors, wherein at least one said acoustic sensor is disposed adjacent to a back region of the body.

37) The method of claim 34 wherein said detecting includes receiving a plurality of electrical signals from a plurality of acoustic sensors, and said acoustic sensors are disposed only adjacent to regions of the body selected from the group consisting of a chest region of the body and a back region of the body.

38) The method of claim 34 wherein said detecting includes receiving a plurality of electrical signals from a plurality of acoustic sensors, and said processing includes computing a parameter indicative of a relation between a said electrical signal and at least one of a history of said electrical signal and a future behavior of said electrical signal.

39) The method of claim 36 wherein said processing includes computing a parameter indicative of a relation between a said electrical signal and at least one of a history of said electrical signal and a future behavior of said electrical signal.

40) The method of claim 38 wherein said processing includes computing a parameter indicative of a relation between a said electrical signal during a first time window and a said electrical signal during a second time window, wherein said first and second time windows overlap.

41) The method of claim 40 wherein said first and second time windows overlap by at least 1 second.

42) The method of claim 41 wherein said first and second time windows overlap by at least 2 seconds.

43) The method of claim 34 wherein said detecting includes receiving a plurality of electrical signals from a plurality of acoustic sensors, and said processing includes computing a parameter indicative of a relation between a first said electrical signal received from a first said acoustic sensor and at least one of a history of said first electrical signal received from said first acoustic sensor and a future behavior of said first electrical signal received from said first acoustic sensor.

44) The method of claim 43 wherein said first acoustic sensor is disposed adjacent to a chest region of the body.

45) The method of claim 43 wherein said first acoustic sensor is disposed adjacent to a back region of the body.

46) The method of claim 38 wherein said relation is indicative of a conditional probability relation.

47) The method of claim 46 wherein said processing includes computing a parameter related to a covariance matrix of said conditional probability relation.

48) The method of claim 46 wherein said processing includes computing at least one eigenvalue of said covariance matrix.

49) The method of claim 48 wherein a lower magnitude of one said eigenvalue is indicative of the one lung ventilation situation.

50) The method of claim 34 wherein said processing includes obtaining a parameter indicative of at least one type of statistics selected from the group consisting of spatial statistics and temporal statistics of said indigenous lung sounds.

51) The method of claim 50 wherein a deviation in said statistics is indicative of a change in an intubation status.

52) The method of claim 34 wherein said processing includes determining a number of distributed random sources of said indigenous lung sounds.

53) The method of claim 52 wherein said processing includes determining only a number of distributed random sources of said indigenous lung sounds.

54) The method of claim 34 wherein said processing includes determining a source scattering parameter indicative of a scattering of noise sources.

55) The method of claim 34 wherein said detecting includes generating an electrical signal, and said processing includes computing an estimate of an autoregressive moving average (ARMA) function of said electronic signal.

56) The method of claim 34 wherein said detecting includes generating an electrical signal, and said processing includes computing an estimate an autoregressive function of said electronic signal.

57) A method of detecting a one lung ventilation situation in a human subject, the method comprising:
a) electronically detecting noise including indigenous lung sounds emanating from a region of the body; and
b) generating an output indicative of the one lung ventilation situation by processing said detected indigenous lung sounds in way that is insensitive to uncancelled, random background noise of a loudness associated with an operating room.

58) The method of claim 57 wherein said uncancelled random background noise includes at least 70 decibels of noise.

59) The method of claim 57 wherein said stage of detecting include detecting noise other than lung sounds, and said stage of processing includes using an adaptive filtering technique to filter noise.

60) A method comprising:
a) selecting a population of human subjects sufficiently large to give statistically significant results; and
b) identifying a one lung intubation situation in a subpopulation of said population,
wherein at most 9.6% of said identifications are misidentifications.

61) A method comprising:
a) selecting a population of human subjects sufficiently large to give statistically significant results; and
b) identifying a one lung intubation situation in a subpopulation of said population,
wherein at most 4.8% of said identifications are false positive identifications, and at most 4.8% of said identifications are false negative identifications.

62) A method comprising:
a) selecting a population of human subjects sufficiently large to give statistically significant results; and
b) identifying a one lung intubation situation in a subpopulation of said population,
wherein at most 9% of said identifications are false positive identifications, and at most 2% of said identifications are false negative identifications.

63) A device for detecting a one lung ventilation situation in a subject, the device comprising:
a) a plurality of acoustic sensors adapted to electronically detect indigenous lung sounds emanating from a region of the body; and
b) a processing unit adapted to process an electronic signal received from said acoustic sensors and to generate an output indicative of a one lung ventilation situation.

64) The device of claim 63 wherein said processing unit is adapted such that at most 9% of said identification are false positive identifications, and at most 2% of said identifications are false negative identifications.

65) The device of claim 63 wherein said processing unit is adapted such that at most 4.5% of said identification are false positive identifications, and at most 4.5% of said identifications are false negative identifications.

66) The device of claim 63 wherein said processing unit is adapted to said output in way that is insensitive to uncancelled, random background noise of a loudness associated with an operating room.
